(19) United States
(12) Patent Application Publication
(10) Pub. No.: US 2025/0053197 A1
(43) Pub. Date: Feb. 13, 2025

(54) DETECTION DEVICE

(71) Applicant: Japan Display Inc., Tokyo (JP)

(72) Inventors: Masahiro TADA, Tokyo (JP); Zhanhao HU, Tokyo (JP)

(21) Appl. No.: 18/797,737

(22) Filed: Aug. 8, 2024

(30) Foreign Application Priority Data
Aug. 9, 2023 (JP) 2023-130265

(51) Int. Cl. G06F 1/16; A61B 5/00; A61B 5/024; H02J 7/00; H02J 50/12; H02J 50/40

(52) U.S. Cl. CPC G06F 1/1632; G06F 1/163; G06F 1/1635; H02J 7/0048; H02J 50/12; A61B 5/02427; A61B 5/6826; H02J 50/40

(57) ABSTRACT

According to an aspect of the present disclosure, a detection device includes: a ring-shaped housing that has a light source, a light sensor, a control circuit, a first power source circuit, and a first battery, the first battery capable of being charged by power from a charger; and a terminal that allows a detachable gadget to be attached detachably to an outside of the housing and enables the detachable gadget to charge the first battery.

### CROSS-REFERENCE TO RELATED APPLICATION

This application claims the benefit of priority from Japanese Patent Application No. 2023-130265 filed on Aug. 9, 2023, the entire contents of which are incorporated herein by reference.

### BACKGROUND

### 1. Technical Field

What is disclosed herein relates to a detection device.

### 2. Description of the Related Art

Devices are known that detect biometric information from human bodies. A ring-shaped authentication device, which is an example of the devices, is disclosed in Japanese Patent Application Laid-open Publication No. 2003-93368. The ring-shaped authentication device is provided with an antenna and receives power carried by radio waves transmitted from an object to be accessed.

The conventional ring-shaped device has electronic components, such as a sensor, a light source, a battery, a controller, and an antenna, in a single housing. Therefore, the ring-shaped housing has a large size, which causes poor wearability. The functions of the conventional ring-shaped device cannot be used while the device is set in the charger. As a result, acquisition of biometric information is interrupted.

For the foregoing reasons, there is a need for a detection device that makes it possible to downsize its ring-shaped housing and to improve its convenience.

### SUMMARY

According to an aspect of the present disclosure, a detection device includes: a ring-shaped housing that has a light source, a light sensor, a control circuit, a first power source circuit, and a first battery, the first battery capable of being charged by power from a charger; and a terminal that allows a detachable gadget to be attached detachably to an outside of the housing and enables the detachable gadget to charge the first battery.

### BRIEF DESCRIPTION OF THE DRAWINGS

FIG. 1 is a schematic diagram illustrating an exemplary appearance in a state in which a finger is placed inside a detection device according to an embodiment;

FIG. 2 is a schematic diagram illustrating an example in which a detachable gadget of the detection device according to the embodiment is detached;

FIG. 3 is a schematic cross-sectional view taken along line A-A in FIG. 2;

FIG. 4 is a block diagram illustrating an exemplary configuration of the detection device;

FIG. 5 is a perspective view illustrating an example of the detachable gadget that can be attached detachably to the detection device;

FIG. 6 is a diagram illustrating an example of charging between the detection device and a charger;

FIG. 7 is a diagram illustrating an exemplary configuration of the charger;

FIG. 8 is a block diagram illustrating an example of charging the detection device;

FIG. 9 is a diagram illustrating an exemplary operation of the detection device;

FIG. 10 is a perspective view illustrating an example of a charging cradle provided with a plurality of chargers; and

FIG. 11 is a schematic cross-sectional view taken along line B-B in FIG. 10.

### DETAILED DESCRIPTION

The following describes an embodiment of the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present specification and the drawings, and detailed description thereof may not be repeated where appropriate.

In the present specification and claims, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### Embodiment

### 1. Exemplary Configuration of Detection Device

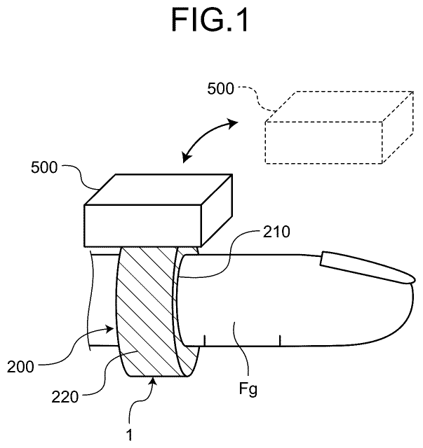

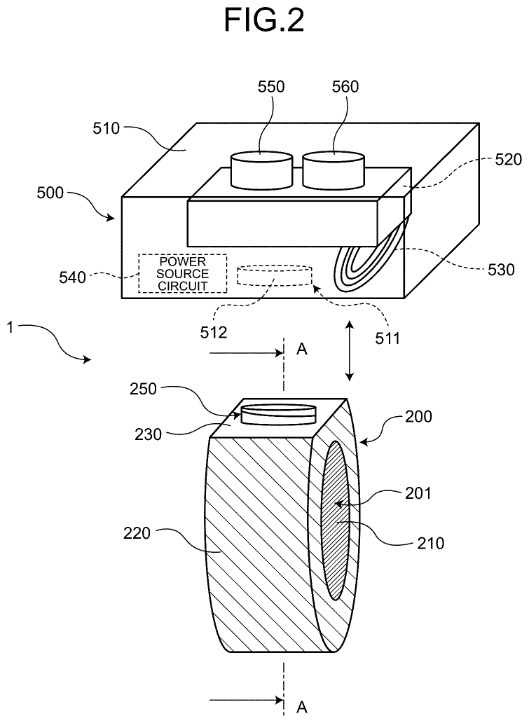

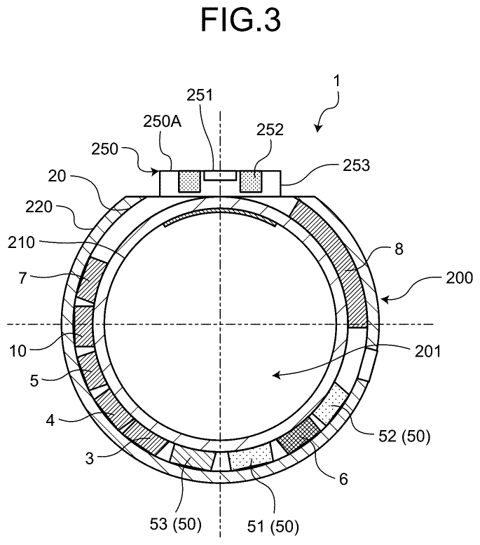

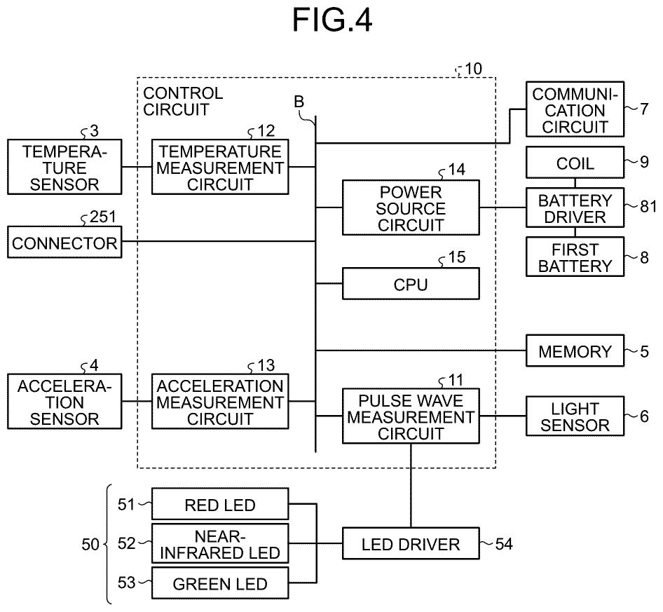

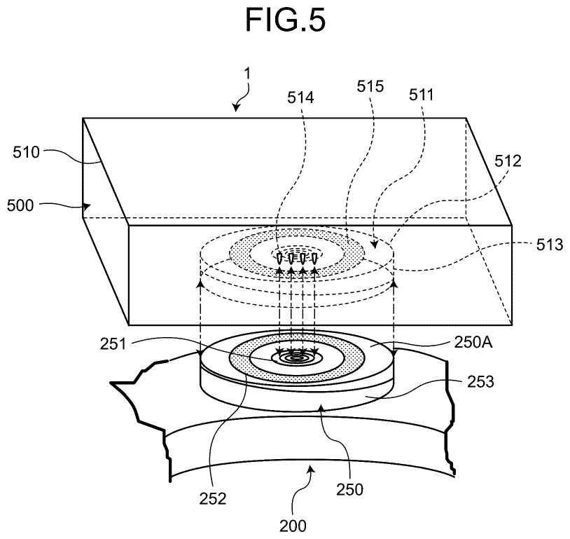

FIG. 1 is a schematic diagram illustrating an exemplary appearance in a state in which a finger is placed inside a detection device according to an embodiment. FIG. 2 is a schematic diagram illustrating an example in which a detachable gadget of the detection device according to the embodiment is detached. FIG. 3 is a schematic cross-sectional view taken along line A-A in FIG. 2. FIG. 3 illustrates a cross-sectional surface of this detection device 1 in FIG. 2 obtained when the detection device 1 is cut by a plane perpendicular to the center line through the center of a hollow of the detection device 1. FIG. 4 is a block diagram illustrating an exemplary configuration of the detection device. FIG. 5 is a perspective view illustrating an example of the detachable gadget that can be attached detachably to the detection device.

The detection device 1 illustrated in FIGS. 1 and 2 is a ring-shaped device that can be attached detachably to a human body and is worn on a finger Fg of the human body. Examples of the finger Fg include a thumb, an index finger, a middle finger, a ring finger, and a little finger. The human body is a person to be authenticated by the detection device 1. The detection device 1 can detect information about a living body, or the human body, (i.e., biometric information) from the finger Fg on which the detection device 1 is worn. The finger Fg is an example of an object to be measured. The object to be measured is the living body or a part of the living body. The detection device 1 is made in a ring or wristband shape, making it easy for a user to carry. In the following description, the detection device 1 is assumed to be used as a ring.

As illustrated in FIG. 2, the detection device 1 includes a ring-shaped housing 200, a terminal 250 by which a detachable gadget 500 is held on the housing 200, and the detachable gadget 500 held by the terminal 250. In the embodiment, in a state in which the housing 200 is worn on the finger Fg, the detection device 1 is capable of being in a first state (refer to FIG. 1) in which the detachable gadget 500 is attached to the housing 200 and a second state (refer to FIG. 2) in which the detachable gadget 500 is detached from the housing 200. The detection device 1 can be used in the first and second states. The detachable gadget 500 supplements the functions of the detection device 1 and serves as a gadget that can charge the detection device 1.

The finger Fg is inserted into a hollow 201 of the housing 200, whereby the housing 200 is worn on the finger Fg while the housing 200 being in contact with the finger Fg. The housing 200 has an inner circumference surface 210 formed of a light-transmissive material, allowing light emitted by a light source 50 housed inside the housing 200 to be transmitted through the inner circumference surface 210 toward the hollow 201. The housing 200 has an outer circumference surface 220 formed of a metal, a non-transparent synthetic resin, or the like that does not allow light emitted by the light source 50 housed inside the housing 200 to be transmitted outward from the outer circumference surface 220. The housing 200 may be configured to expose a light sensor 6 from the inner circumference surface 210 or to house the light sensor 6 near the inner circumference surface 210.

The terminal 250 is formed in a cylindrical projection shape projected from the outer circumference surface 220 of the housing 200. In the embodiment, the housing 200 has a contact section 230, which is a flattened section of the outer circumference surface 220, such that the contact section 230 can contact the outer surface of the detachable gadget 500. The terminal 250 is formed as a projection projected from the central section of the contact section 230 of the housing 200. This allows the detection device 1 to increase the contact area between the housing 200 and the detachable gadget 500, thereby enhancing the stability of the housing 200 and the detachable gadget 500 when the detachable gadget 500 is attached to the housing 200 without increasing the size of the terminal 250.

As illustrated in FIG. 3, the housing 200 has a temperature sensor 3, an acceleration sensor 4, a memory 5, the light sensor 6, a communication circuit 7, a first battery 8, a control circuit 10, the light source 50, and a connector 251. These components are each mounted on a flexible substrate 20. These components can exchange signals with each other via the flexible substrate 20. The housing 200 houses therein the temperature sensor 3, the acceleration sensor 4, the memory 5, the light sensor 6, the communication circuit 7, the first battery 8, the control circuit 10, and the light source 50.

A plurality of types of light emitting diodes (LEDs) can be used for the light source 50. In the embodiment, the light source 50 has a red LED 51, a near-infrared LED 52, and a green LED 53. The red LED51 outputs red light. The near-infrared LED 52 outputs near-infrared light. The green LED 53 outputs green light. The light source 50 operates an LED driver 54, which is not illustrated, to drive the red LED 51, the near-infrared LED 52, and the green LED 53 to emit light therefrom.

The light sensor 6 detects light that is emitted by the light source 50 and reflected by the finger Fg and the like, and light directly incident thereon, etc. The light sensor 6 is an organic photodiode (OPD). The light sensor 6 receives light that is output from the red LED 51, the near-infrared LED 52, and the green LED 53 and passes through the finger Fg inserted in the hollow 201. The light sensor 6 detects a change in intensity of the received light. The light sensor 6, which is an organic photodiode (OPD), for example, outputs an electrical signal corresponding to the received light.

When the red LED 51 is lit, and the near-infrared LED 52 and the green LED 53 are not lit, the red light passing through the finger is input to the light sensor 6. When the near-infrared LED 52 is lit, and the red LED 51 and the green LED 53 are not lit, the near-infrared light passing through the finger is input to the light sensor 6. When the green LED 53 is lit, and the red LED 51 and the near-infrared LED 52 are not lit, the green light passing through the finger is input to the light sensor 6.

The light emitted from the light source 50 is reflected by the surface of the object to be detected, such as the finger Fg, and enters the light sensor 6. This allows the detection device 1 to detect a fingerprint by detecting the shape of ridges and valleys on the surface of the finger Fg and the like. Alternatively, the light emitted from the light source 50 may be reflected inside the finger Fg and the like, or transmitted through the finger Fg and the like, and then incident on the light sensor 6. This allows the detection device 1 to detect the biometric information about the living body inside the finger Fg and the like. Examples of the biometric information include the pulse waves, pulsations, and vascular image of the finger or a palm. That is, the detection device 1 may be configured as a fingerprint detection device to detect the fingerprint, or a vein detection device to detect a vascular pattern of, for example, veins.

The temperature sensor 3 detects the temperature of the finger Fg when the finger Fg is inserted into the hollow 201. The temperature sensor 3 detects the temperature of the environment surrounding the housing 200 when the finger Fg is not inserted into the hollow 201. The temperature sensor 3 provides information indicating the detected temperature to the control circuit 10.

The acceleration sensor 4 detects the acceleration applied to the detection device 1. The acceleration sensor 4 provides information indicating the detected acceleration to the control circuit 10. The detected acceleration value can be used to remove the effects of body motion of the wearer of the detection device 1.

The communication circuit 7 has an antenna for short-range wireless communication, the antenna being not illustrated. The communication circuit 7 exchanges signals between the detection device 1 and other devices. The communication circuit 7 can transmit data measured by the various components of the detection device 1 to other devices, for example. The communication circuit 7 can also receive data transmitted by other devices, for example. Examples of other devices include portable devices, such as smartphones and tablets, carried by the user of the detection device 1. The portable device, such as the smartphone and tablet, has a display screen. The data received from the detection device 1 is displayed on the display screen, allowing the user of the detection device 1 to see the data.

The first battery 8 is a secondary battery, for example. The first battery 8 is a chemical battery that can be used while being repeatedly charged and discharged. The examples of the first battery 8 include a storage battery, and a rechargeable battery. The first battery 8 is compatible with Qi (an international standard for wireless power transfer), for example. The first battery 8 can supply the stored power to each of the components and the like that require power in the detection device 1. The first battery 8 is electrically coupled to each of the temperature sensor 3, the acceleration sensor 4, the light sensor 6, the communication circuit 7, the control circuit 10, and the light source 50 to supply power to each of those components.

The control circuit 10 controls each component of the detection device 1. The control circuit 10 is an integrated circuit (IC), such as a microcontroller. The control circuit 10 may be a programmable logic device (PLD) such as a field programmable gate array (FPGA).

As illustrated in FIG. 4, the control circuit 10 has a pulse wave measurement circuit 11, a temperature measurement circuit 12, an acceleration measurement circuit 13, a power source circuit 14, and a central processing unit (CPU) 15. The circuits are coupled by a bus B and can exchange data with each other via the bus B. The control circuit 10 is electrically coupled to the memory 5 and the communication circuit 7.

The memory 5 is a storage unit that stores therein various types of data. Example aspects of the memory 5 may include a random access memory (RAM), a read only memory (ROM), and an electrically erasable programmable read only memory (EEPROM). The memory 5 stores therein various types of information used by the control circuit 10. The memory 5 may be included in the control circuit 10.

The pulse wave measurement circuit 11 is coupled to the LED driver 54 and the light sensor 6. The pulse wave measurement circuit 11 measures a pulse frequency, a blood oxygen concentration, etc. based on the data detected by the light sensor 6. The LED driver 54 drives the red LED 51, the near-infrared LED 52, and the green LED 53 to emit light therefrom.

The temperature measurement circuit 12 is electrically coupled to the temperature sensor 3. The temperature measurement circuit 12 receives temperature data from the temperature sensor 3.

The acceleration measurement circuit 13 is electrically coupled to the acceleration sensor 4. The acceleration measurement circuit 13 receives acceleration data from the acceleration sensor 4.

The power source circuit 14 is coupled to a battery driver 81. The battery driver 81 is electrically coupled to the first battery 8 and a coil 9. The coil 9 is a charging coil for charging the first battery 8 and has a winding wound along the inner circumference surface 210 of the housing 200. The housing 200 houses the coil 9 in the vicinity of the inner circumference surface 210 and allows a magnetic field to pass through the housing 200. The coil 9 is electrically coupled to a rectifier circuit, which is not illustrated. When approaching a power transmission coil of a charger or the like, the coil 9 magnetically couples with the power transmission coil and receives an electromagnetic field from the power transmission coil to convert the electromagnetic field into an electric current. The coil 9 may also be used as a near field communication (NFC) antenna to capture electromagnetic waves in space and absorb energy. The power source circuit 14 controls the charging of the first battery 8 and supplies power from the first battery 8 to each component.

The CPU 15 is the control unit that controls each component in the control circuit 10. The CPU 15 executes a predetermined program to measure or calculate the biometric information such as a pulse wave velocity, a blood pressure, and the pulse frequency.

The CPU 15 supplies control signals to the pulse wave measurement circuit 11, the temperature measurement circuit 12, and the acceleration measurement circuit 13 to control detection operations. The CPU 15 stores, in the memory 5, the measurement results of each circuit, the biometric information based on those measurement results, etc. The CPU 15 provides the control signal for the light source 50 to the LED driver 54 to control the turning on and off of the light source 50.

As illustrated in FIG. 3, the connector 251 is provided to the terminal 250 of the housing 200 and is electrically coupled to the detachable gadget 500 attached to the housing 200. The connector 251 is used to exchange data, power, etc. with the detachable gadget 500. The connector 251 can be formed using a four-concentric ring electrode, for example.

As illustrated in FIG. 5, the terminal 250 has the connector 251 disposed at or near the center of a surface 250A and a magnet 252 disposed to surround the connector 251. The terminal 250 has a thread formed on a side wall 253 from the surface 250A to the housing 200. The connector 251 supplies power supplied from the detachable gadget 500 to the power source circuit 14, achieving charging of the first battery 8 from the detachable gadget 500. The connector 251 supplies power from the power source circuit 14 to the detachable gadget 500. The detection device 1, thus, allows mutual power supply between the housing 200 and the detachable gadget 500.

The detachable gadget 500 has a second housing 510 that can be attached detachably to the housing 200. The second housing 510 is formed of a synthetic resin, a metal, or the like in a cuboid shape, for example. The second housing 510 has a cylindrical recess 511 into which the terminal 250 of the housing 200 is fitted. The detachable gadget 500 has a connector 514 disposed at or near the center of a bottom 512 of the recess 511 and a magnet 515 disposed to surround the connector 514. The connector 514 is a pogo pin connector, for example. The pogo pin has a plunger, a spring, a barrel, etc. The connector 514 is electrically coupled to a power source circuit 540 and supplies power from an external source to the power source circuit 540 to charge the second battery 520. The second housing 510 has a thread on a side surface 513. The thread on the side surface 513 screws with the thread on the side wall 253 of the terminal 250 of the housing 200 such that the ridges of the thread on the side wall 253 are engaged in the grooves of the thread on the side surface 513. When the detachable gadget 500 is attached to or detached from the housing 200, the detachable gadget 500 moves in the direction of attachment to or removal from the housing 200, while rotating with the side surface 513 and the side wall 253 of the terminal 250 engaged. In this case, the connector 514 rotates along a circular groove concentric with the connector 251. When the terminal 250 of the housing 200 is fitted into the recess 511 of the detachable gadget 500, the connector 251 of the terminal 250 and the connector 514 are electrically coupled, and the magnet 252 of the terminal 250 and the magnet 515 are attracted to each other. Screwing the terminal 250 of the housing 200 into the recess 511 of the detachable gadget 500 increases the attachment strength between the housing 200 and the detachable gadget 500 when the detachable gadget 500 is attached to the housing 200.

As illustrated in FIG. 2, the second housing 510 accommodates the second battery 520, a second coil 530, the power source circuit 540, a light emitting device 550, and a haptic element (haptic device) 560. Each of these components is mounted on a substrate, which is not illustrated, and is housed in the second housing 510. When the detachable gadget 500 is attached to the housing 200, each of the components in the second housing 510 is electrically coupled to the control circuit 10 of the housing 200 and can be operated under the control of the control circuit 10.

The second battery 520, which is a lithium ion battery, for example, supplies power to each component of the detachable gadget 500. The second battery 520 supplies power for charging the first battery 8 of the housing 200 when the detachable gadget 500 is attached to the housing 200. Alternatively, the second battery 520 is charged with power supplied from the housing 200. The second coil 530 for receiving power is electrically coupled to the second battery 520. When the second coil 530 approaches the power transmission coil, the second coil 530 magnetically couples with the power transmission coil to receive the electromagnetic field from the power transmission coil. The received electromagnetic field is converted into an electric current. As a result, the second battery 520 can be charged with the electric current.

The power source circuit 540 is electrically coupled to the second battery 520 and the second coil 530. The second coil 530 is a charging coil for being charged by a charger and has a winding wound along the surface of the second housing 510. The second housing 510 houses the second coil 530 in the vicinity of the inner circumference surface 210 and allows a magnetic field to pass through the second housing 510. The second coil 530 is electrically coupled to a rectifier circuit, which is not illustrated. When approaching a power transmission coil of a charger or the like, the second coil 530 magnetically couples with the power transmission coil and receives an electromagnetic field from the power transmission coil to convert the electromagnetic field into an electric current. The second coil 530 may also be used as an NFC antenna to capture electromagnetic waves in space and absorb energy. The power source circuit 540 controls the charging of the second battery 520 and supplies power from the second battery 520 to each component.

The light emitting device 550, which is an LED, is disposed on the surface, inside, or the like of the second housing 510 and capable of making a notification of various types of information about the detection device 1. The light emitting device 550 makes a notification of an alert under the control of the control circuit 10 of the housing 200. A plurality of light emitting devices 550 may be provided to the second housing 510.

The haptic device 560, which is disposed inside the second housing 510, applies vibration to the second housing 510 to vibrate the housing 200 having the detachable gadget 500 attached thereto. The control circuit 10 of the housing 200 controls the haptic device 560 to start and end the application of vibration. This allows the detection device 1 to transfer vibrations or the like to the finger Fg when the housing 200 is worn on the finger Fg, without providing, in the housing 200, a configuration for the vibration.

In the embodiment, the light emitting device 550 and the haptic device 560 achieve the notification function of the detection device 1 outside the housing 200. This allows the detection device 1 to contribute to downsize the housing 200, because it is not necessary to provide, in the housing 200, a configuration for notification.

The detachable gadget 500 includes the second housing 510 and the recess 511 (mounting section). The housing 510 accommodates the second battery 520, the second coil 530, the light emitting device 550, and the haptic device 560. The recess 511 allows the second housing 510 to be attached detachably to the outer circumference surface 220 of the ring-shaped housing 200. The detachable gadget 500 thus can be provided as a gadget that charges the first battery 8 of the housing 200 via the recess 511 with the power of the second battery 520.

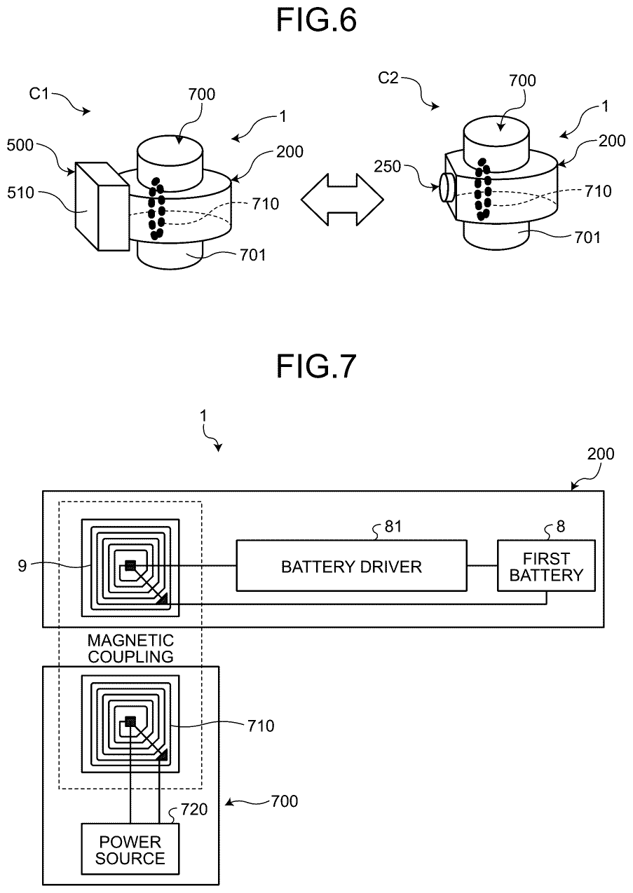

FIG. 6 is a diagram illustrating an example of charging between the detection device 1 and a charger 700. In the embodiment, the detection device 1 may include the charger 700 that can charge the first battery 8 in the housing 200 in a wireless manner from the outside of the housing 200. As illustrated in a case C1 in FIG. 6, the detection device 1 can charge the first battery 8 from the charger 700 in the first state in which the detachable gadget 500 is attached. As illustrated in a case C2 in FIG. 6, the detection device 1 can charge the first battery 8 from the charger 700 in the second state in which the detachable gadget 500 is not attached. The charger 700 is formed to have a cylindrical third housing 701 with an outer diameter slightly smaller than the inner diameter of the housing 200 of the detection device 1. The charger 700 has an engagement portion, which is not illustrated, formed on the outer periphery of the third housing 701, which engages and positions the detection device 1 such that a third coil 710 provided in the charger 700 faces the coil 9 of the detection device 1.

FIG. 7 is a diagram illustrating an exemplary configuration of the charger 700. As illustrated in FIG. 7, the charger 700 includes the third coil 710 that can supply power to the coil 9 of the detection device 1 and a power source 720 that can supply power to the third coil 710. The third coil 710 and the power source 720 are electrically coupled via a drive circuit 711. The third coil 710, which is a resonance type coil, is operated by a drive voltage from the power source 720. The power source 720 is an alternating current (AC) power source. The charger 700 is a device that provides power in a wireless manner by magnetically coupling the third coil 710 with the coil 9 of the housing 200, which functions as a power receiving coil and is in proximity to the charger 700.

The exemplary configuration of the detection device 1 according to the embodiment is described above. The configuration described with reference FIGS. 1 to 7 is only an example. The configuration of the detection device 1 according to the embodiment is not limited to the example. The configuration of the detection device 1 according to the embodiment can be flexibly modified depending on specifications and operations thereof.

### 2. Exemplary System Configuration of Detection Device

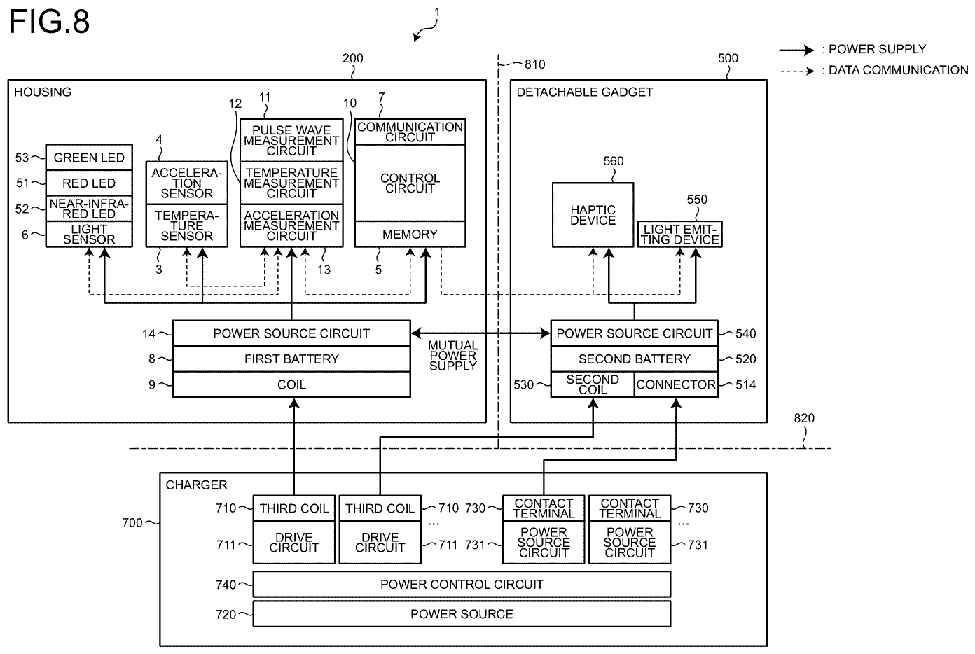

FIG. 8 is a block diagram illustrating an example of charging the detection device 1. In FIG. 8, the solid arrows indicate power supply and the dashed arrows indicate data communication. As illustrated in FIG. 8, the detection device 1 includes the detachable gadget 500 and the charger 700.

The detection device 1 is configured such that the temperature sensor 3, the acceleration sensor 4, the memory 5, the light sensor 6, the communication circuit 7, the control circuit 10, the pulse wave measurement circuit 11, the temperature measurement circuit 12, the acceleration measurement circuit 13, the light source 50, etc. are supplied with power from the first battery 8 of the housing 200. The control circuit 10 of the housing 200 can perform data communication with the pulse wave measurement circuit 11, the temperature measurement circuit 12, the acceleration measurement circuit 13, etc. The detection device 1 is configured such that the light emitting device 550, the haptic device 560, etc. are supplied with power from the second battery 520 of the detachable gadget 500.

The charger 700 includes a plurality of third coils 710, the power source 720, a plurality of contact terminals 730, and a power control circuit 740. The third coils 710 are each arranged to correspond to one of the coil 9 of the housing 200 and the second coil 530 of the detachable gadget 500. The third coils 710 are electrically coupled to the respective drive circuits 711, and each of the third coils 710 is operated by the corresponding drive circuit 711 with a drive voltage from the power source 720. The contact terminals 730 are arranged in such a manner to be able to contact the connector 514 and the like of the detachable gadget 500. The contact terminals 730 are electrically coupled to respective power source circuits 731, and each of the contact terminals 730 supplies power from the power source 720 via the corresponding power source circuit 731.

The detection device 1 allows mutual power supply and data communication between the housing 200 and the detachable gadget 500 using a first interface 810. The detection device 1 allows each of the housing 200 and the detachable gadget 500 to be charged by the charger 700 using a second interface 820.

The control circuit 10 of the housing 200 can perform control by data communication with the light emitting device 550, the haptic device 560, etc. of the detachable gadget 500 attached to the housing 200 via the connector 251.

The housing 200 can provide power in a wireless manner by magnetically coupling the coil 9 with the third coils 710 of the charger 700. When the connector 251 is electrically coupled with the connector 514 of the detachable gadget 500, the housing 200 can receive power from and supply power to the detachable gadget 500 via the connector 251.

For example, as illustrated in FIG. 1, when the detachable gadget 500 is attached to the housing 200 and the housing 200 is not fixed to the charger 700, the first battery 8 of the detection device 1 can be charged with power supplied from the detachable gadget 500 via the connector 251.

For example, as illustrated in the case C1 in FIG. 6, the detection device 1 charges the first battery 8 with power in a wireless manner by the magnetic coupling between the coil 9 and the third coils 710 of the charger 700, when the housing 200 is attached to the detachable gadget 500 and the housing 200 is fixed to the charger 700. In this case, when the residual amount of the second battery 520 of the detachable gadget 500 is equal to or below a determination threshold, power is supplied from the first battery 8 or the charger 700 to the detachable gadget 500 by the control circuit 10 of the housing 200 in the detection device 1. The detection device 1 thus can charge the second battery 520 of the detachable gadget 500 with power from the housing 200.

For example, as illustrated in the case C2 in FIG. 6, when the housing 200 is fixed to the charger 700 and the detachable gadget 500 is not attached to the housing 200, the first battery 8 of the detection device 1 is charged with power in a wireless manner by the magnetic coupling between the coil 9 and the third coils 710 of the charger 700.

### 3. Exemplary Operation of Detection Device

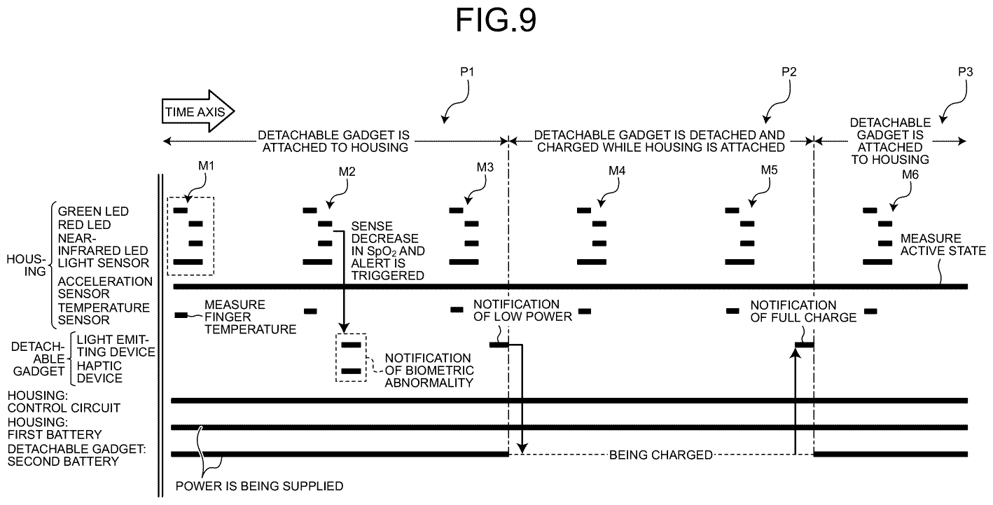

FIG. 9 is a diagram illustrating an exemplary operation of the detection device 1. FIG. 9 illustrates the operation periods of time based on the basic configuration of the housing 200 and the detachable gadget 500 in the detection device 1. The basic configuration is as follows: the housing 200 includes the light source 50, the light sensor 6, the acceleration sensor 4, the temperature sensor 3, the control circuit 10, and the first battery 8; and the detachable gadget 500 includes the light emitting device 550, the haptic device 560, and the second battery 520.

As illustrated in FIG. 9, the detection device 1 periodically performs, using power from the first battery 8 of the housing 200, measurement in measurement periods M1, M2, M3, M4, M5, and M6, in each of which the green LED 53 is lit for a predetermined time, then the red LED 51 and the near-infrared LED 52 are lit for a predetermined time, and light emitted from each of the LEDs is measured by the light sensor 6. In the example illustrated in FIG. 9, the measurements in the measurement periods M1, M2, and M3 are performed in a period P1 in which the detachable gadget 500 is attached to the housing 200. The measurements in the measurement periods M4 and M5 are performed in a period P2 in which the detachable gadget 500 is detached from the housing 200 and charged, and only the housing 200 is worn on the finger Fg. The measurement in the period M6 is performed in a period P3 in which the detachable gadget 500 after being charged is attached to the housing 200.

The detection device 1 constantly operates the acceleration sensor 4 and the control circuit 10 using the power of the first battery 8 of the housing 200 and sequentially performs the measurements in the measurement periods M1, M2, M3, M4, M5, and M6 in which the detection device 1 periodically operates the temperature sensor 3. In the detection device 1, each component to be operated is supplied with power from the first battery 8 of the housing 200.

In the periods P1 and P3 in which the detachable gadget 500 is attached to the housing 200, the detection device 1 is in a state in which the second battery 520 of the detachable gadget 500 is allowed to supply power to the light emitting device 550 and the haptic device 560 or the first battery 8 of the housing 200.

In the detection device 1 in the period P1, the control circuit 10 detects a decrease in SpO₂ based on the measurement result in the measurement period M2, and sends notification information notifying a biometric abnormality to the light emitting device 550 and the haptic device 560 of the detachable gadget 500, thereby causing the light emitting device 550 to be turned on (lit) and the haptic device 560 to be vibrated. This allows the detection device 1 to notify the user of the biometric abnormality by the lighting of the light emitting device 550 and the vibration of the haptic device 560 of the detachable gadget 500.

Thereafter, when the measurement period M3 of the detection device 1 ends and the control circuit 10 detects that the second battery 520 of the detachable gadget 500 has low power, the control circuit 10 sends notification information for notification of the low power to the light emitting device 550, thereby causing the light emitting device 550 to flash. The term “low power” means that the residual amount of power of the battery is equal to or below a predetermined value. The detection device 1 thus can make the user aware that the second battery 520 of the detachable gadget 500 has low power by flashing the light emitting device 550 of the detachable gadget 500.

In the period P2, the user detaches the detachable gadget 500 from the housing 200 and charges the second battery 520 of the detachable gadget 500 using the charger 700. In this case, the housing 200 from which the detachable gadget 500 is detached performs the periodic measurements in the measurement periods M4 and M5. Afterward, when the detachable gadget 500 is fully charged, the detachable gadget 500 is attached to the housing 200 in the detection device 1. When the control circuit 10 of the detection device 1 detects that the second battery 520 of the detachable gadget 500 is fully charged, the control circuit 10 sends notification information for notification of a fully charged amount to the light emitting device 550, thereby causing the light emitting device 550 to be turned on. The detection device 1 thus can make the user aware that the second battery 520 of the detachable gadget 500 is fully charged by the lighting of the light emitting device 550 of the detachable gadget 500 attached to the housing 200. The first battery 8 of the housing 200 in the detection device 1 may be charged with power from the second battery 520 of the detachable gadget 500 attached to the housing 200.

The operation of the detection device 1 then proceeds from the period P2 to the period P3. In the period P3, in the same manner as the period P1, the detection device 1 performs the measurement in the measurement period M6 in which the green LED 53 is lit for a predetermined time, then the red LED 51 and the near-infrared LED 52 are lit for a predetermined time, and the light emitted from each of the LEDs is measured by the light sensor 6, using the power from the first battery 8 in the housing 200. Thereafter, the detection device 1 periodically repeats the measurement.

As described above, the detection device 1 includes: the ring-shaped housing 200 that has the light source 50, the light sensor 6, the control circuit 10, the power source circuit 14 (first power source circuit), and the first battery 8, the first battery 8 capable of being charged with power from the charger 700; and the terminal 250 that allows the detachable gadget 500 to be attached detachably to the outside of the housing 200 and enables the detachable gadget 500 to charge the first battery 8. The detection device 1 thus can charge the first battery 8 of the housing 200 using the charger 700 and charge the first battery 8 of the housing 200 from the detachable gadget 500 attached to the outside of the housing 200, thus allowing charging the first battery 8 while the housing 200 is worn by a user, thereby downsizing the first battery 8 to be housed in the housing 200. The detection device 1 can share the battery between both the housing 200 and the detachable gadget 500, thereby making it possible to make the housing 200 thinner and to improve the wearability of the detection device 1. The detection device 1 can be used while the detachable gadget 500 is detached, allowing continuous use of the detection device 1. As a result, the detection device 1 can downsize the ring-shaped housing 200 and improve its convenience.

In the detection device 1, the detachable gadget 500 includes the second battery 520 and the power source circuit 540 (the second power source circuit). When the detachable gadget 500 is attached to the terminal 250, the second battery 520 of the detachable gadget 500 and the first battery 8 of the housing 200 can be charged by each other via the power source circuit 540 of the detachable gadget 500. The detection device 1 thus can charge the first battery 8 from the detachable gadget 500 attached to the outside of the housing 200 and charge the second battery 520 of the detachable gadget 500 from the first battery 8. As a result, the detection device 1 can charge the second battery 520 of the detachable gadget 500 when charging the first battery 8 of the housing 200, thereby improving the convenience.

In the detection device 1, the detachable gadget 500 includes the second coil 530 to receive power for charging the second battery 520 from the third coils 710 (external coil) of the charger. The detection device 1 thus can charge the detachable gadget 500 singly in a wireless manner, thereby further reducing the opportunity to remove the housing 200 from the finger Fg. As a result, the detection device 1 allows the user to wear the housing 200 for a long period of time, thereby reducing the period during which the housing 200 is not worn for charging.

In the detection device 1, the detachable gadget 500 includes at least one of the light emitting device 550 and the haptic device 560 that can be controlled by the control circuit 10 of the housing 200 when the detachable gadget 500 is attached to the terminal 250 of the housing 200. The detection device 1 thus can downsize the housing 200 because at least one of the light emitting device 550 and the haptic device 560 does not need to be housed in the housing 200. As a result, the detection device 1 can downsize the housing 200 and improve the wearability and enhance the functions of the detection device 1.

In the detection device 1, the light source 50 emits at least one of visible light, near-infrared light, and green light, and the control circuit 10 detects information about the living body that the housing 200 contacts, on the basis of information detected by the light sensor 6. The detection device 1 thus can cause the housing 200 to be worn for a long period of time, thereby reducing the interruption of the detected information about the living body.

In the detection device 1, the housing 200 has at least one of the temperature sensor 3 and the acceleration sensor 4. The detection device 1 thus can cause the housing 200 to be worn for a long period of time, thereby reducing the interruption of the information about the detected temperature, acceleration, etc.

In the detection device 1, at least one of the light emitting device 550 and the haptic device 560 of the detachable gadget 500 makes notification of the state of the second battery 520 of the detachable gadget 500. With this notification of the state of the second battery 520, the second battery 520 can be charged quickly.

The detection device 1 further includes the charger 700 that can charge the first battery 8 of the housing 200 in a wireless manner from the outside of the housing 200. With this configuration, the first battery 8 of the detection device 1 can be charged in a wireless manner by the charger 700 even when the housing 200 is downsized, thereby preventing the reduction in the convenient.

### Modification of Embodiment

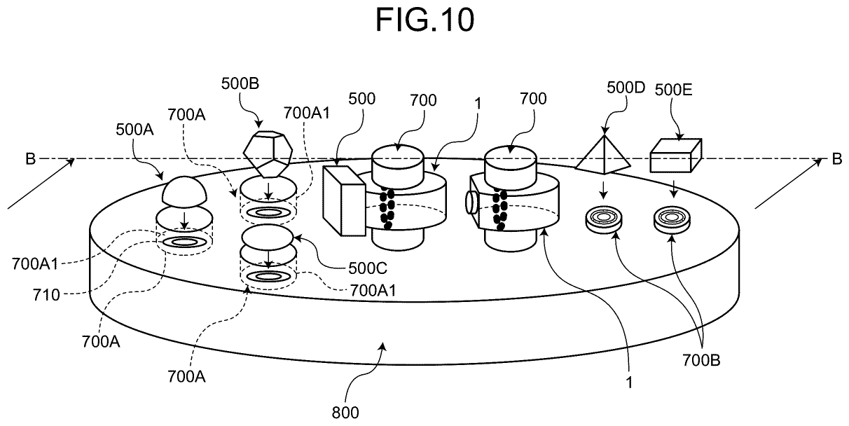

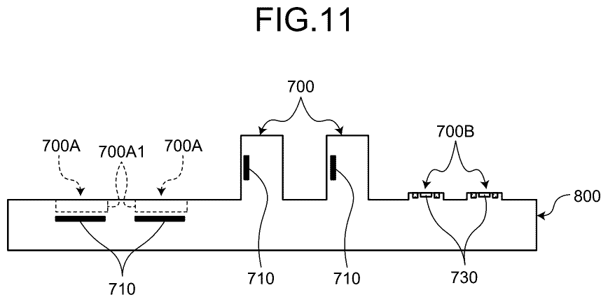

FIG. 10 is a perspective view illustrating an example of a charging cradle provided with a plurality of chargers. FIG. 11 is a schematic cross-sectional view taken along line B-B in FIG. 10. As illustrated in FIGS. 10 and 11, this charging cradle 800 is formed in a disk shape and has a plurality of the chargers 700, a plurality of chargers 700A, and a plurality of chargers 700B. In the example illustrated in FIG. 10, the charging cradle 800 has the two chargers 700, the three chargers 700A, and the two chargers 700B.

The charger 700A charges, in a non-contact manner, detachable gadgets 500A, 500B, and 500C that can be attached detachably to the detection device 1. The detachable gadgets 500A, 500B, and 500C have the same configuration as the detachable gadget 500, but only differ from the detachable gadget 500 in the shape of the second housing 510. The housing of the detachable gadget 500A has a semi-spherical shape. The housing of the detachable gadget 500B has a polyhedral shape. The housing of the detachable gadget 500C has an oval shape. As illustrated in FIGS. 10 and 11, each of the chargers 700A has a recess 700A1 into which one of the detachable gadgets 500A, 500B, 500C, etc. is fitted, and a third coil 710 provided on the bottom of the recess 700A1. The third coil 710 is electrically coupled to the power source 720 described above. Each of the chargers 700A is a device that provides power in a wireless manner by magnetically coupling the third coil 710 with the second coil 530 of one of the detachable gadgets 500A, 500B, 500C, etc. being fitted into the recess 700A1.

As illustrated in FIG. 10, each of the chargers 700B charges one of detachable gadgets 500D and 500E, which can be attached detachably to the detection device 1, via the connector. The detachable gadgets 500D and 500E each have the same configuration as the detachable gadget 500, but only differ from the detachable gadget 500 in the shape of the second housing 510. The housing of the detachable gadget 500D has a square pyramid shape. The housing of the detachable gadget 500E has a cube shape. As illustrated in FIGS. 10 and 11, each of the chargers 700B is formed in a protruding shape and includes the contact terminal 730 as described above. Power from the power source 720 is supplied to the contact terminal 730. Each of the chargers 700B is a device that supplies power to one of the detachable gadgets 500D, 500E, etc. via the contact between the contact terminal 730 and the connector 514 of the detachable gadget.

The charging cradle 800 can be provided with the chargers 700, the chargers 700A, and the chargers 700B having different charging methods, making it possible to charge the first battery 8 of the housing 200 of the detection device 1 and the detachable gadgets 500, 500A, 500B, 500C, 500D, 500E, etc. having various shapes. The detection device 1 thus can use the detachable gadgets 500 and the like having various shapes, thereby improving the product value.

The embodiment describes the configuration in which the detection device 1 includes the detachable gadget 500 and the charger 700. The configuration is not limited to this configuration. The detection device 1 may include any one of the detachable gadget 500 and the charger 700. The detection device 1 may be a system that includes a device having the configuration of the housing 200, the detachable gadget 500, and the charger 700.

The above mentioned embodiment can be combined with each component as appropriate. Other operational advantages accruing from the aspects described in the present embodiment that are obvious from the description herein, or that are conceivable as appropriate by those skilled in the art will naturally be understood as accruing from the present disclosure.

What is claimed is:

1. A detection device comprising:
a ring-shaped housing that has a light source, a light sensor, a control circuit, a first power source circuit, and a first battery, the first battery capable of being charged by power from a charger; and
a terminal that allows a detachable gadget to be attached detachably to an outside of the housing and enables the detachable gadget to charge the first battery.

2. The detection device according to claim 1, further comprising the detachable gadget configured to be attached to the terminal, wherein
the detachable gadget includes:
a second battery; and
a second power source circuit, and
the second battery of the detachable gadget and the first battery of the housing are capable of being charged by each other via the second power source circuit when the detachable gadget is attached to the terminal.

3. The detection device according to claim 2, wherein the detachable gadget includes a second coil to receive power for charging the second battery from an external coil.

4. The detection device according to claim 2, wherein the detachable gadget includes at least one of a light emitting device and a haptic device that are capable of being controlled by the control circuit of the housing when the detachable gadget is attached to the terminal.

5. The detection device according to claim 4, wherein
the light source emits at least one of visible light, near-infrared light, and green light, and
the control circuit detects information about a living body that the housing contacts, based on information detected by the light sensor.

6. The detection device according to claim 5, wherein the housing has at least one of a temperature sensor and an acceleration sensor.

7. The detection device according to claim 6, wherein at least one of the light emitting device and the haptic device of the detachable gadget makes notification of a state of the second battery.

8. The detection device according to claim 7, further comprising the charger that is capable of charging the first battery of the housing in a wireless manner from an outside of the housing.
